(19) United States
(12) Patent Application Publication
(10) Pub. No.: US 2026/0165879 A1
(43) Pub. Date: Jun. 18, 2026

(54) OPHTHALMIC SURGICAL SYSTEM FOR TREATING AN EYE

(71) Applicant: CARL ZEISS MEDITEC AG, Jena (DE)

(72) Inventors: Max Wagenblast, Oberkochen (DE); Christoph Kuebler, Oberkochen (DE); Alexander Schenk, Oberkochen (DE)

(21) Appl. No.: 19/417,309

(22) Filed: Dec. 11, 2025

(30) Foreign Application Priority Data
Dec. 12, 2024 (DE) 10 2024 137 508.6

(51) Int. Cl. A61F 9/007

(52) U.S. Cl. CPC A61F 9/00736

(57) ABSTRACT

An ophthalmic surgical system for treating an eye and a method for operating the ophthalmic surgical system are provided. The ophthalmic surgical system is configured to detect an occlusion at a needle tip, determine intraocular pressure increase information which describes a target intraocular pressure increase by which a predetermined target intraocular pressure in the eye to be treated should be increased, by applying an increase determination algorithm, determine a control command for at least one irrigation-side fluid pump by applying a command determination algorithm to the determined intraocular pressure increase information, with the determined control command predetermining an increase in a current intraocular pressure to a sum of the target intraocular pressure and the target intraocular pressure increase by supplying treatment fluid into the eye, and control the least one irrigation-side fluid pump at least until an occlusion break is detected in accordance with the determined control command.

### CROSS REFERENCE TO RELATED APPLICATIONS

This application claims priority to German patent application DE 10 2024 137 508.6, filed Dec. 12, 2024, the entire content of which is incorporated herein by reference.

### TECHNICAL FIELD

The disclosure relates to an ophthalmic surgical system for treating an eye. The disclosure also relates to a method for operating such an ophthalmic surgical system for treating a lens of an eye.

### BACKGROUND

An ophthalmic surgical system serves for treating an eye of a patient, for example within the scope of a cataract operation, in which the eye of the patient has a lens opacification, which is referred to as a cataract or grey star. Phacoemulsification is particularly widespread in this context; here, a thin needle, in particular a hollow needle, is introduced into a capsular bag of the eye to be treated and is excited to perform mechanical vibrations within the capsular bag. The needle, which is introduced into the eye in the region of a needle tip of the needle, is part of an ophthalmic surgical handpiece. The lens of the eye is comminuted with the aid of the vibrating needle tip, whereby lens particles are released in the eye. The released lens particles are aspirated via an aspiration line of the ophthalmic surgical system. At the same time, the eye is supplied with irrigation fluid via an irrigation line of the ophthalmic surgical system. As soon as the lens has been completely removed from the eye, a new artificial lens can be inserted into the emptied capsular bag. For example, the treated patient might achieve improved vision compared to their vision prior to the operation. In an alternative or in addition, the ophthalmic surgical system may be configured for use within the scope of a vitrectomy, in which a vitreous humor in the eye is comminuted with the ophthalmic surgical handpiece.

A situation in which a lens particle blocks the aspiration line of the ophthalmic surgical system may arise during a use of the ophthalmic surgical system. Such a situation may be referred to as an occlusion of the needle tip of the handpiece of the ophthalmic surgical system. An increase in the intraocular pressure in the eye of the patient is observed immediately after the occurrence of the occlusion since the eye is still supplied with the irrigation fluid via the irrigation line. However, aspiration-side suctioning from the eye is prevented by the lens particle that caused the occlusion.

The lens particle blocking the aspiration line can be comminuted with the needle, which for example carries out vibrations in the ultrasonic wave range, whereupon the blockage of the aspiration line can be ended. This event may be referred to as occlusion break. After the occlusion break, an aspiration fluid with the lens particles can be abruptly aspirated from the eye via the aspiration line. This leads to an abrupt reduction in pressure in the eye, i.e., a fast and sudden reduction in the intraocular pressure and in an anterior chamber volume in the eye. However, an eye collapse, for example, may occur if the intraocular pressure, or the anterior chamber volume in the eye, is reduced too much. The sudden reduction in the intraocular pressure following an occlusion break should thus at least be mitigated.

WO 2022/219527 A1 discloses a surgical cassette serving for an ophthalmic surgical system and comprising an irrigation system and an aspiration system. An aspiration pump in the aspiration system generates a vacuum pressure within an aspiration line in order to transport a fluid from the eye. In the case of an occlusion, a tube of the aspiration line may be modified from a larger cross-sectional area to a smaller cross-sectional area. The smaller cross-sectional area may be maintained during increased pumping via the aspiration line observed after an occlusion break. There may subsequently be a switch back to the larger cross-sectional area.

WO 2019/069204 A2 discloses a system for detecting an intraocular pressure event during a phacoemulsification. In the case of an occlusion event, an intraocular pressure in an eye of a patient may be increased by virtue of increasing an infusion pressure.

### SUMMARY

It is an object of the disclosure to provide a solution with which an intraocular pressure drop in an eye of a patient after an occlusion break can be reduced.

The object is achieved by an ophthalmic surgical system and a method for operating the ophthalmic surgical system as described herein.

A first aspect of the disclosure relates to an ophthalmic surgical system for treating an eye of a patient. The ophthalmic surgical system includes at least one console which includes at least one portion of at least one irrigation-side fluid pump for conveying a treatment fluid and at least one portion of at least one aspiration-side fluid pump for conveying an aspiration fluid. Alternatively, the treatment fluid may be referred to as irrigation fluid. In a typical example, the ophthalmic surgical system includes two irrigation-side fluid pumps and two aspiration-side fluid pumps.

Moreover, the ophthalmic surgical system includes an ophthalmic surgical handpiece for treating the eye. At least a portion of the ophthalmic surgical handpiece is introducible into the eye with a needle tip of the handpiece. The ophthalmic surgical handpiece is coupled at least indirectly to the at least one irrigation-side fluid pump and the at least one aspiration-side fluid pump.

A needle-tip-including needle of the handpiece is configured as a hollow needle such that the aspiration fluid can be aspirated from the eye and the treatment fluid can be pumped into the eye through the needle. In this context, the treatment fluid is for example guided into the eye via a sleeve that surrounds the hollow needle. The sleeve has a passage opening with a diameter which is larger than an outer diameter of the hollow needle and from which the hollow needle projects. In this case, the treatment fluid is guided into the eye via a fluid channel which is formed between an outer side of the hollow needle and an inner side of the sleeve, and which ends at the passage opening. Alternatively, the sleeve includes a separate opening for the treatment fluid in addition to the passage opening. This sleeve can be introduced into the eye together with the hollow needle in the region of the needle tip. The ophthalmic surgical handpiece includes two fluid lines that are separated from each other, one for the treatment fluid and one for the aspiration fluid, and so two mutually separate hydraulic connections to the eye of the patient may be formed.

For example, the console may include a cassette accommodation region for accommodating a cassette. In this case, the at least one irrigation-side fluid pump and/or the at least one aspiration-side fluid pump may be at least partially included by the cassette. For example, the cassette may be connected directly to the ophthalmic surgical handpiece via a supply device. In an alternative exemplary embodiment, the console may be connected directly to the ophthalmic surgical handpiece via the supply device. The supply device for example includes at least one portion of an aspiration line for the aspiration fluid and at least one portion of an irrigation line for the treatment fluid. The handpiece thus includes two lines that are separate from each other.

In other words, the ophthalmic surgical system includes an irrigation side and an aspiration side. On the irrigation side, the at least one irrigation-side fluid pump can be used to pump the treatment fluid from a treatment fluid storage vessel, which may be arranged outside the console of the ophthalmic surgical system, to the handpiece and, from the latter, into the eye of the patient. Furthermore, on the aspiration side, the aspiration fluid can be pumped out of the eye to an aspiration fluid collection vessel via the handpiece with the at least one aspiration-side fluid pump. Alternatively, the aspiration fluid collection vessel may be referred to as drainage container.

In particular, the ophthalmic surgical system is configured to detect an occlusion at the needle tip by applying an occlusion detection algorithm. An occlusion is an event or a situation in which at least one access to the aspiration line of the ophthalmic surgical system is obstructed at the needle tip of the handpiece. For example, a lens particle in the eye may at least temporarily close and thus block an entrance of the aspiration line encompassed by the handpiece. The occlusion detection algorithm includes at least one rule and/or instruction which, when carried out, detects the occlusion, for example on the basis of measurement data provided by the ophthalmic surgical system. In this context, an example allows for the use of known methods for detecting an occlusion at the needle tip of a handpiece.

The ophthalmic surgical system is configured to determine intraocular pressure increase information by applying an increase determination algorithm following the detection of the occlusion. The intraocular pressure increase information describes a target intraocular pressure increase by which a predetermined target intraocular pressure of the eye to be treated is to be increased, i.e., should be increased. The target intraocular pressure increase thus describes a desired increase in the intraocular pressure of the eye to be treated, in relation to a target value for the intraocular pressure, which is referred to here as target intraocular pressure. For example, the target intraocular pressure is specified, in particular in a manner specific to the patient, by a treating person, such as a surgeon or any other member of medical staff, before the start of or during the treatment of the eye. In an alternative or in addition, the target intraocular pressure may be determined and provided, in particular be predetermined automatically, with the ophthalmic surgical system. The target intraocular pressure may alternatively be referred to as a set intraocular pressure (IOP) value. The predetermined target intraocular pressure is the intraocular pressure that should typically be maintained at a constant level during the treatment of the eye and hence during the use of the ophthalmological system. Here, the assumption is made that the ophthalmological system is typically operated during the occlusion in such a way that controlling the at least one irrigation-side fluid pump sets the target intraocular pressure in the eye. For example, should the intraocular pressure initially rise after the onset of the occlusion since treatment fluid is still supplied to the eye without aspiration fluid being able to be aspirated, appropriate control of the at least one irrigation-side fluid pump causes the intraocular pressure to be reduced again until it has reached the target intraocular pressure, and the latter is subsequently kept constant. However, a different control of the at least one irrigation-side fluid pump is sought according to the disclosure; in this case, the target intraocular pressure is increased by the target intraocular pressure increase and temporarily kept at an increased level.

The increase determination criterion includes at least one instruction and/or rule, the implementation of which causes the calculation and hence determination of the intraocular pressure increase information. As a result of applying the increase determination criterion, the determined intraocular pressure increase information depends on a current treatment situation. The increase determination criterion thus takes into account at least one piece of information describing the current treatment situation. The current treatment situation may depend on the ophthalmic surgical system used, for example on the handpiece used, and/or on the patient treated. Thus, when the increase determination criterion is applied, there is a treatment-situation-specific and hence situation-specific determination of the intraocular pressure increase information, and no predetermined value for the target intraocular pressure increase is assumed. Hence the desired increase for the intraocular pressure is determined dynamically.

The disclosure also includes an ophthalmic surgical system which is configured to determine absolute intraocular pressure information by applying the increase determination criterion, the absolute intraocular pressure information describing the intraocular pressure to which the intraocular pressure in the eye should be increased, for example starting from the target intraocular pressure or a current intraocular pressure. In an alternative to the target intraocular pressure increase or in addition, it is thus already possible to calculate the sum of the target intraocular pressure and target intraocular pressure increase and hence calculate an intraocular pressure target that should be set in the eye during the occlusion.

The ophthalmological system is configured to determine a control command for the at least one irrigation-side fluid pump by applying a command determination algorithm to the determined intraocular pressure increase information. The command determination algorithm for example includes at least one instruction and/or rule, the implementation of which on the basis of the determined intraocular pressure increase information causes the control command for the at least one irrigation-side fluid pump to be calculated and hence determined. The determined control command specifies an increase of a current intraocular pressure to a sum of the target intraocular pressure and target intraocular pressure increase by supplying treatment fluid into the eye. The control command is thus suitable for allowing the at least one irrigation-side fluid pump to be controlled in accordance therewith. However, the control command is only provided for temporarily controlling the at least one irrigation-side fluid pump during the occlusion. Should the at least one irrigation-side fluid pump be controlled in accordance with the control command, the intraocular pressure in the eye of the patient can be increased in a targeted manner to a value that can be calculated from the predetermined target intraocular pressure and the target intraocular pressure increase in accordance with the determined intraocular pressure increase information. For example, the current intraocular pressure is detected or calculated with a sensor of the ophthalmic surgical system and therefore assumed as known. In an exemplary embodiment, the calculation of the current intraocular pressure may include estimating the current intraocular pressure.

The ophthalmic surgical system is configured to control the at least one irrigation-side fluid pump in accordance with the determined control command at least until an occlusion break of the detected occlusion is detected. The occlusion break of the detected occlusion can be detected by applying an occlusion break detection algorithm. The occlusion break detection algorithm may correspond at least in part to the occlusion detection algorithm or may differ from the latter. For example, the occlusion break detection algorithm includes at least one rule and/or instruction, the implementation of which causes the break and hence an end of the occlusion detected previously to be ascertained. Thus, the determined control command is implemented with the ophthalmic surgical system, i.e., the intraocular pressure is increased at least up to the occlusion break. In particular, control in accordance with the control command in this context does not assume that the intraocular pressure in the eye must initially correspond to the target intraocular pressure; instead, the intraocular pressure may be increased to the target intraocular pressure increased by the target intraocular pressure increase from any desired current intraocular pressure.

Thus, the occlusion is detected with the ophthalmological system according to the disclosure, whereupon a desired target intraocular pressure increase and, depending thereon, a control command for the irrigation-side fluid pump are determined, with the determined control command being implemented with the irrigation side such that the intraocular pressure is increased in accordance with the determined target intraocular pressure increase until the occlusion break is detected. What is achieved as a result is that, depending on the current treatment situation which may relate both to the ophthalmological system itself and to the patient to be treated, a drop in the intraocular pressure occurring after the occlusion break, for example, is at least attenuated since the intraocular pressure had previously been increased. For example, what this at least achieves is that, for example, a minimal intraocular pressure, which should set in in the eye as a minimum, cannot be reached following the occlusion break in order to prevent damage to the eye. Thus, what is achieved is that an intraocular pressure drop in an eye of a patient following an occlusion break can be reduced in comparison with typical ophthalmic surgical systems.

An exemplary embodiment provides for the ophthalmological system to be configured to determine an expected volume flow of aspiration fluid by applying the increase determination algorithm, in accordance with which expected volume flow of aspiration fluid aspiration fluid is expected to the removed, i.e., aspirated, from the eye with the at least one aspiration-side fluid pump after the occlusion break. Thus, the expected volume flow from the eye of the patient after the time of the occlusion break is calculated. In that case, a volume of aspiration fluid is likely to be removed, and this can be determined by multiplying the expected volume flow by a time period, with the time period being at least as long as the sum of a detection time for detecting the occlusion break and an algorithm latency which for example takes into account a delay time as a result of applying the occlusion break detection algorithm and a response time of the irrigation side to the occlusion break. To this end, the increase determination algorithm for example includes at least one rule and/or instruction, on the basis of which the expected volume flow is calculated. The determined volume flow is a calculated volume flow; in other words, this means an estimated or predicted volume flow which need not necessarily correspond to the actual volume flow which is aspirated from the eye after the occlusion break. In an exemplary embodiment, the expected volume flow may at least substantially correspond to the actual volume flow. For example, substantially encompasses deviations of up to 1 percent, 2 percent, 3 percent, 5 percent or 10 percent in particular.

Application of the increase determination algorithm further leads to the ophthalmological system being configured to determine a target anterior chamber volume increase in the eye taking into account the determined expected volume flow. Said target anterior chamber volume increase in the eye describes a desired anterior chamber volume increase in the eye before the occlusion break, i.e., during the occlusion, in relation to an anterior chamber volume in the eye of an anterior chamber in the eye of the patient before the occlusion. In other words, the target anterior chamber volume increase in the eye is determined in relation to the original anterior chamber volume in the eye before the occlusion. This procedure is based at least on the insight that in the event of an elevated intraocular pressure, a volume of an anterior chamber in the eye of the patient, i.e., an anterior chamber volume in the eye, is increased at the same time, in each case in relation to any initial situation which is characterized by an initial intraocular pressure and an initial anterior chamber volume in the eye assigned to the initial intraocular pressure.

Moreover, the ophthalmic surgical system is configured to determine an intraocular pressure assigned to the determined target anterior chamber volume increase in the eye in a predetermined correspondence curve by applying the increase determination algorithm. The predetermined correspondence curve describes a relationship between the anterior chamber volume increase in the eye and the intraocular pressure. For example, the predetermined correspondence curve may be provided as a lookup table. The entries in the lookup table may be recorded as a diagram in which an intraocular pressure is plotted on an x-axis and the anterior chamber volume increase in the eye is plotted on a y-axis, or vice versa. Now, the value in the correspondence curve, in particular in the lookup table, assigned to the determined target anterior chamber volume increase in the eye is read off. In other words, an intraocular pressure is read off the correspondence curve taking into account the determined target anterior chamber volume increase in the eye. The intraocular pressure read off is referred to as the determined assigned intraocular pressure.

The ophthalmic surgical system is configured to determine an intraocular pressure difference between the predetermined target intraocular pressure and the determined assigned intraocular pressure by applying the increase determination algorithm. For example, the intraocular pressure difference can be determined by subtracting the target intraocular pressure from the determined assigned intraocular pressure. The intraocular pressure difference may alternatively be referred to as intraocular pressure discrepancy. The determined intraocular pressure difference describes or provides a numerical value for the target intraocular pressure increase described by the intraocular pressure increase information. In other words, the intraocular pressure difference determined as described above is assumed to be the target intraocular pressure increase, on the basis of which the control command is determined.

It becomes clear that the manner in which the anterior chamber volume in the eye should be changed by increasing the intraocular pressure is determined taking into account an expected outflow of aspiration fluid following an occlusion break. This is a particularly suitable procedure for being able to determine the intraocular pressure increase information in treatment-situation-specific fashion since the volume flow depends at least on the ophthalmic surgical system used, for example the handpiece used, but also on the predetermined target intraocular pressure and hence on the eye of the patient.

For example, the predetermined correspondence curve is stored, in particular saved, in a memory device of the ophthalmic surgical system. For example, the predetermined correspondence curve has been calculated from numerous data records which describe a relationship between the anterior chamber volume increase in the eye and the intraocular pressure for numerous patients.

A further exemplary embodiment includes that the ophthalmic surgical system is configured to determine a pressure difference between the predetermined target intraocular pressure and a current negative pressure in the at least one aspiration-side fluid pump in order to determine the expected volume flow. The pressure difference may alternatively be referred to as pressure discrepancy. For example, the current negative pressure can be subtracted from the predetermined target intraocular pressure. The current negative pressure sets in on the aspiration side if an aspiration fluid flow is prevented on account of the occlusion but at the same time work is carried out with, e.g., maximum pumping power of the at least one aspiration-side fluid pump. Thus, in a typical exemplary embodiment, the current negative pressure is a maximum negative pressure or minimum pressure that can be set or is set on the aspiration side. For example, the current negative pressure can be determined, in particular measured, with a pressure sensor in the at least one aspiration-side fluid pump.

For example, if the at least one aspiration-side fluid pump is configured as a diaphragm pump, in which a separation element separates a pump chamber from a drive chamber, it is possible to generate the current negative pressure in the diaphragm pump, for example by aspirating a drive fluid, in particular air, from the drive chamber, without aspiration fluid being able to be sucked into the pump chamber at the same time on account of the occlusion. The current negative pressure also illustrates why comparatively large amounts of aspiration fluid are quickly pumped out of the eye following the occlusion break, as a result of which the intraocular pressure is reduced quickly and significantly.

The ophthalmic surgical system is configured to determine, in a predetermined further correspondence curve which describes a relationship between the volume flow and an aspiration-side pressure drop, an assigned volume flow assigned to the pressure drop corresponding to the determined pressure difference in order to determine the expected volume flow. The determined assigned volume flow describes the expected volume flow which, as described above, may be subject to further evaluation in order to determine the intraocular pressure increase information. The further correspondence curve does not correspond to the above-described correspondence curve. For example, the further correspondence curve describes the volume flow on an x-axis and the aspiration-side pressure drop on a y-axis, or vice versa. The aspiration-side pressure drop is equated with the previously determined pressure difference such that the volume flow assigned to the determined pressure difference is read off from the further correspondence curve and assumed to be the expected volume flow.

For example, the predetermined further correspondence curve is stored, in particular saved, in the memory device of the ophthalmic surgical system. For example, the predetermined further correspondence curve has been calculated from at least one data record which describes a relationship between pressure drop and volume flow for at least one handpiece.

The further correspondence curve may be predetermined specifically for the handpiece in particular. For example, a profile of the correspondence curve may depend on a diameter of the needle tip of the handpiece. For example, a larger pressure drop for the same volume flow may be expected if the diameter of the needle tip is smaller than in the case of a different handpiece. In a typical example, the handpiece used is verified before the use of the ophthalmic surgical system and at least before the detection of the occlusion. Depending on the handpiece ascertained within the scope of the verification, it is then possible to provide at least the further correspondence curve, for example by virtue of loading the latter from the memory device. The described determination of the expected volume flow is particularly specific to the current treatment situation on account of the considered negative pressure and the further correspondence curve, and so it may contribute to determining the suitable intraocular pressure increase information.

Another exemplary embodiment provides for the ophthalmic surgical system, when determining the target anterior chamber volume increase in the eye, to be configured to take into account, in addition to the determined expected volume flow, a predetermined time period required for detecting the occlusion break and a response of the ophthalmic surgical system to the detected occlusion break. In particular, said ophthalmic surgical system is configured to multiply the determined expected volume flow by the predetermined time period, with the product obtained in this case describing the target anterior chamber volume increase in the eye.

According to an exemplary embodiment, the predetermined time period is composed of a pure sensory detection time for detecting the occlusion break, a delay time due to the application of the occlusion break detection algorithm and a response time of the irrigation side to the occlusion break. In this exemplary embodiment, the response time is a time that is required to set the target intraocular pressure again by supplying treatment fluid. In an exemplary embodiment, the sensory detection time, for example, may be 200 milliseconds. The detection time for example depends on an overall tube length of the ophthalmic surgical system and/or a speed at which a pressure wave generated in the event of an occlusion break or an effect of the pressure wave can be detected by sensory means with the at least one aspiration-side fluid pump. The delay time due to the application of the occlusion break detection algorithm may be understood to be a delay time due to the algorithm. It is 30 milliseconds, for example. The response time of the irrigation side is 300 milliseconds, for example. Other detection, delay and/or response times are possible. The predetermined time period thus is a parameter that depends on the ophthalmic surgical system used. Ultimately, what is achieved by taking into account the predetermined time period is that, taking into account the expected volume flow, it is possible to determine for how long how much aspiration fluid is aspirated until the occlusion break can be detected, and there can be a response thereto on the irrigation side. The volume of aspiration fluid aspirated during the predetermined time period may be understood to be the product of the predetermined time period and the expected volume flow. This leads to an accurate determination of the target anterior chamber volume increase in the eye.

In a further exemplary embodiment, the ophthalmic surgical system is configured to additionally take into account a predetermined safety factor in order to determine the target anterior chamber volume increase in the eye. The predetermined safety factor for example specifies a tolerance range with which, e.g., internal inaccuracies in the setting of the intraocular pressure and/or inaccuracies in the algorithms used and/or in the used sensors of the ophthalmic surgical system can be taken into account. The safety factor may be 1, for example, or for example be increased to 1.1 or 1.2. Higher or lower safety factors are possible. In particular, they may be predetermined depending on the ophthalmic surgical system used, the medical staff, the treated patient and/or other boundary conditions. In an exemplary embodiment, the predetermined safety factor is multiplied by the determined expected volume flow and the predetermined time period.

A typical exemplary embodiment provides for the ophthalmic surgical system to be configured to adopt a conformity curve, which may alternatively be referred to as compliance curve or standard curve, as the predetermined correspondence curve which describes the relationship between the anterior chamber volume increase in the eye and the intraocular pressure. For example, a description of a conformity curve can be found in the publication “Effect of a new phacoemulsification and aspiration handpiece on anterior chamber stability” by Suzuki, Hisaharu; Igarashi, Tsutomu; Takahashi, Hiroshi (Journal of cataract and refractive surgery, 49(1), pages 91-96, 2023).

For example, the conformity curve may be obtained from numerous data records of patients, for example several thousand data records. Thus, the conformity curve is for example a correspondence curve averaged over numerous patients. In an alternative, a patient curve determined in a patient-specific manner may be adopted. For example, the latter may be selected from a list of numerous conformity curves that were determined for different patients. For example, the patient curve may be selected from the list of numerous conformity curves on the basis of an eye size, in particular an anterior chamber volume in the eye, of the patient. In an alternative to that, a personalized patient curve, for example, may be determined, in particular measured, for the patient. In this example, the correspondence curve may be predetermined on an individual basis for each patient. In an alternative, a group curve which is predetermined for a patient group is adopted. In particular, the patient group depends on at least one of a sex, an age, and/or an ethnicity, i.e., an ethnic group. The group curve is based at least on the insight that age, sex, and/or ethnicity may have an influence on the intraocular pressure of the patient.

By choosing the predetermined correspondence curve, it is possible to select and predetermine a degree of personalization chosen for the determination of the intraocular pressure increase information. For example, use may initially be made of the conformity curve, and there may be a switch to group curves and/or patient curves after a few operations and data records recorded in the process.

Another exemplary embodiment provides for the ophthalmic surgical system to be configured to determine new intraocular pressure increase information at least once after the control of the at least one irrigation-side fluid pump in accordance with the determined control command and before the occlusion break is detected. In this case, a target intraocular pressure adapted to a response time of the eye to a change in the intraocular pressure is taken into account instead of the predetermined target intraocular pressure. This adapted target intraocular pressure may deviate from the predetermined target intraocular pressure or substantially correspond thereto. How setting of the predetermined target intraocular pressure with the at least one irrigation-side fluid pump can be implemented is taken into account. For example, if the predetermined target intraocular pressure is set slowly in comparison with other patients and/or another handpiece, for example on account of an elasticity of an eyeball of the eye of the patient, then it is possible to take into account a reduced target intraocular pressure. However, if the predetermined target intraocular pressure for example is set quickly in comparison with other patients and/or another handpiece, then it is possible to take into account an elevated target intraocular pressure or the predetermined target intraocular pressure.

The ophthalmic surgical system is configured to determine a new control command by applying the command determination algorithm to the determined new intraocular pressure increase information and to control the at least one irrigation-side fluid pump at least until the detection of the occlusion break in accordance with the new actuation command. This procedure may be performed repeatedly a number of times if the occlusion persists, and so for example multiple new pieces of intraocular pressure increase information and multiple new control commands may may be determined in succession and the latter may be implemented. This procedure is based at least on the insight that a driving pressure discrepancy in the ophthalmic surgical system is increased as a result of an already elevated intraocular pressure after control in accordance with the control command determined first. For example, this may result in the occurrence of a slightly higher volume flow after the occlusion break, which is why a further calculation of the intraocular pressure increase information and ultimately of the control command may be expedient. As a result, it is ultimately possible to determine and implement the target intraocular pressure increase iteratively with increased computational outlay but even more accurately overall.

A different exemplary embodiment provides for the ophthalmic surgical system to be configured to follow the determination of the intraocular pressure increase information with carrying out a check as to whether the latter lies in a predetermined intraocular pressure value range. The control command is determined should the check be resolved in the affirmative, in particular only if said check is resolved in the affirmative. Thus, for example, a maximum value and a minimum value for the intraocular pressure increase may be predetermined. For example, the minimum value may be chosen such that a predetermined minimum increase in the intraocular pressure is ensured. For example, this may be chosen on the basis of a diameter of the needle tip, in particular in such a way that an anterior chamber collapse in the eye can be ruled out. For example, the maximum value may be chosen in such a way that it is possible to rule out the attainment of an undesirably strong increase in the intraocular pressure, in the case of which, for example, endothelial cells in the eye or an optic nerve of the eye might possibly be damaged. Furthermore, both the lower and the upper limit value, and hence the maximum value and the minimum value, may be set to be the same, in particular in order for example to be able to set a constant intraocular pressure increase independently of the current treatment situation. Provision may be made for the predetermined intraocular pressure value range to depend on the predetermined target intraocular pressure, i.e., be predetermined differently for different target intraocular pressures. Ultimately, a check is carried out as to whether the determined intraocular pressure increase information is reasonable and unable to lead to an endangerment of the patient. This leads to a safeguard with regard to unreasonable intraocular pressure increases, which might possibly be incorrectly determined by the ophthalmic surgical system.

Furthermore, an exemplary embodiment provides for the ophthalmic surgical system to be configured to apply the occlusion detection algorithm to movement information in order to detect the occlusion. The movement information describes a current movement of a separation element between the pump chamber and the drive chamber of at least one of the fluid pumps, in particular of the at least one aspiration-side fluid pump. For example, the movement information is determined with a position sensor associated with the fluid pump in question. In that case, the occlusion is detected at least on the basis of the movement information being smaller than a predetermined first movement limit value. The first movement limit value may be predetermined in such a way that the separation element no longer moves or substantially no longer moves. In this case, substantially is intended to encompass deflections of the separation element of up to 1 percent, 2 percent, 3 percent, 5 percent, or 10 percent, in relation to a maximum possible deflection. The lack of a separation element movement may thus indicate that the occlusion has occurred since, for example, no aspiration-side movement of the separation element can be observed during said occlusion.

In an alternative or in addition, the ophthalmic surgical system is configured to apply the occlusion break detection algorithm to movement information in order to detect the occlusion break and detect the occlusion break at least on the basis of the movement information being larger than a predetermined second movement limit value. In this case, the at least one aspiration-side fluid pump may be considered. Thus, whenever a movement in the at least one fluid pump is ascertained, the inference is made that the occlusion has broken, and the ophthalmic surgical system is capable of pumping aspiration fluid out of the eye again.

The two movement limit values mentioned here, i.e., the first movement limit value and the second movement limit value, may be same or different from each other. For example, the predetermined first movement limit value for detecting the occlusion may be chosen to be smaller than the predetermined second movement limit value for detecting the occlusion break, or vice versa. Only the at least one aspiration-side fluid pump is considered in a typical example.

Thus, the occlusion and the occlusion break can be detected reliably and quickly in this way of the basis of the separation element movement, and the information gathered in the process can be processed reliably and quickly as well. As soon as the occlusion break has been detected, the determined control command is typically withdrawn again or no longer accepted, and the ophthalmic surgical system is controlled in such a way that the intraocular pressure is regulated to the predetermined target intraocular pressure and thus set.

A further aspect of the disclosure relates to a method for operating an ophthalmic surgical system, having at least: a console which includes at least one portion of at least one irrigation-side fluid pump for conveying a treatment fluid and at least one portion of at least one aspiration-side fluid pump for conveying an aspiration fluid, and an ophthalmic surgical handpiece, at least a portion of said handpiece being introducible into the eye with a needle tip of the handpiece and said handpiece being coupled at least indirectly to the irrigation-side fluid pump and the aspiration-side fluid pump. In an exemplary embodiment, the method is performed during treatment of an eye, for example during cataract surgery or vitreous humor surgery, i.e., a vitrectomy. The method should not be understood as a treatment method but as a method which performs calculations in order determine and implement a control command for the ophthalmic surgical system.

In particular, the method includes detecting an occlusion at the needle tip by applying an occlusion detection algorithm. Intraocular pressure increase information is determined following the detection of the occlusion, said information describing a target intraocular pressure increase by which a predetermined target intraocular pressure, for example in the eye to be treated, should be increased, by applying an increase determination algorithm, whereby the determined intraocular pressure increase information depends on a current treatment situation. A control command for the at least one irrigation-side fluid pump is determined by applying a command determination algorithm to the determined intraocular pressure increase information, with the determined control command predetermining an increase in a current intraocular pressure to a sum of the target intraocular pressure and the target intraocular pressure increase by supplying treatment fluid into the eye. The least one irrigation-side fluid pump is controlled at least until an occlusion break of the detected occlusion is detected by applying an occlusion break detection algorithm in accordance with the determined control command.

For example, the method is performed with a control apparatus of the ophthalmic surgical system. For example, the control apparatus includes a processor device. The latter may include at least one microprocessor, microcontroller, a field programmable gate array (FPGA) and/or a digital signal processor (DSP).

The exemplary embodiments described in connection with the ophthalmic surgical system according to aspects of the disclosure, in each case individually and in combination with one another, apply, mutatis mutandis and if applicable, to the method according to an aspect of the disclosure. The disclosure includes combinations of the exemplary embodiments described.

### BRIEF DESCRIPTION OF THE DRAWINGS

The disclosure will now be described with reference to the drawings wherein:

FIG. 1 shows a schematic illustration of an ophthalmic surgical system;

FIG. 2 shows a schematic illustration of fluid flows in an ophthalmic surgical system prior to an occlusion;

FIG. 3 shows a schematic illustration of fluid flows within an ophthalmic surgical system during an occlusion;

FIG. 4 shows a schematic illustration of the flow rates on the irrigation side and aspiration side before and during an occlusion;

FIG. 5 shows a schematic illustration of a typical profile of an irrigation-side and aspiration-side fluid pump pressure or control air pressure before, during and after an occlusion;

FIG. 6 shows a schematic illustration of a profile of the intraocular pressure according to a standard procedure and according to an exemplary embodiment of the disclosure;

FIG. 7 shows a schematic illustration of a signal flow graph for a method for operating an ophthalmic surgical system;

FIG. 8 shows a schematic illustration of a signal flow graph of details from the method according to FIG. 7;

FIG. 9 shows a schematic illustration of a correspondence curve;

FIG. 10 shows a schematic illustration of a further correspondence curve; and

FIG. 11 shows a schematic illustration of a fluid pump of an ophthalmic surgical system.

### DESCRIPTION OF EXEMPLARY EMBODIMENTS

In the figures, functionally identical elements are given the same reference numerals.

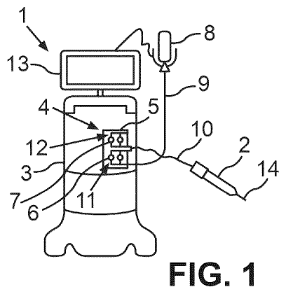

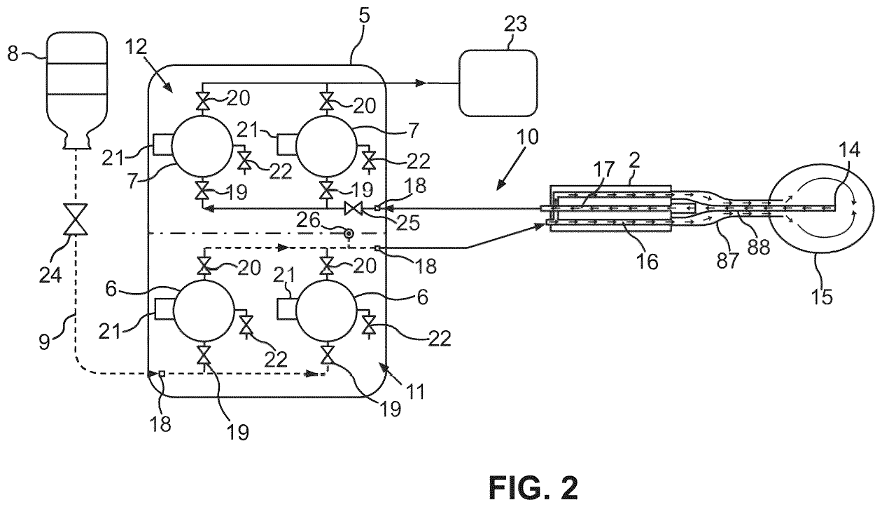

FIG. 1 shows an ophthalmic surgical system 1. The ophthalmic surgical system 1 includes an ophthalmic surgical handpiece 2 which is suitable, with a needle tip 14, for insertion into an eye 15 (see reference numeral 15 in FIG. 2) of a patient to be treated within the scope of an operation, for example a cataract operation. The ophthalmic surgical system 1 includes a console 3. The latter for example includes a cassette accommodation region 4 for a cassette 5 of the ophthalmic surgical system 1. The console 3 and/or the cassette 5 for example includes at least one portion of at least one irrigation-side fluid pump 6 for conveying a treatment fluid 16 (see reference numeral 16 in FIG. 2), the latter for example being able to be provided in a treatment fluid storage vessel 8 and be guided via a treatment fluid line 9 to the console 3 or to the cassette 5. The console 3 and/or the cassette 5 includes for example at least one portion of at least one aspiration-side fluid pump 7 for conveying an aspiration fluid 17 (see reference numeral 17 in FIG. 2).

There may be a supply device 10 present between the console 3 or the cassette 5 and the ophthalmic surgical handpiece 2, said supply device for example allowing the treatment fluid 16 to be guided to the handpiece 2 and/or allowing the aspiration fluid 17 to be guided from the handpiece 2 to the console 3 or cassette 5. The cassette 5 may therefore include, for example, an irrigation side 11 for pumping the treatment fluid 16 in the direction of the handpiece 2 and an aspiration side 12 for aspirating the aspiration fluid 17 from the eye 15.

The ophthalmic surgical system 1 may further include a display device and/or user interface 13, which for example can display information to a person treating the patient.

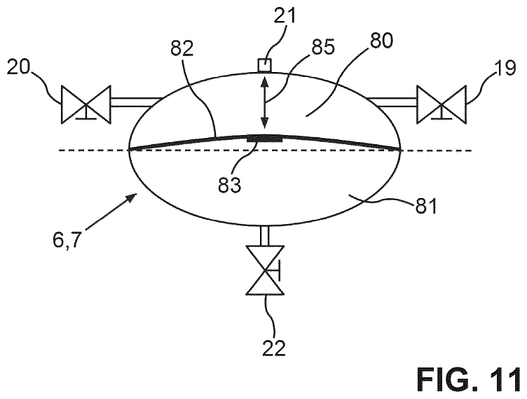

FIG. 2 shows fluid flows within the ophthalmic surgical system 1 before occlusion. It becomes clear that the handpiece 2 is inserted into the eye 15 of the patient with the needle tip 14 while the ophthalmic surgical system 1 is used for treating the eye 15. Moreover, details of the cassette 5 and of the fluid pumps 6, 7 encompassed thereby are shown as an example. It becomes clear that numerous valves 19, 20, 22 may be arranged in the cassette 5 both on the irrigation side 11 and on the aspiration side 12. Respective inlet valves 19 for admitting into the respective fluid pump 6, 7 and outlet valves 20 for discharging the treatment fluid 16 and aspiration fluid 17, respectively, from the respective fluid pump 6, 7 are shown in this case. Furthermore, a compressed-air valve 22 for a compressed-air supply into the respective fluid pump 6, 7 may be present. A deflection 85 (see reference numeral 85 in FIG. 11) and hence a movement of a separation element 82 (see reference numeral 82 in FIG. 11) of the respective fluid pump 6, 7 may be detected with a position sensor 21. Furthermore, a pressure sensor 26 may be provided in the cassette 5. Provision can be made for an aspiration fluid collection vessel 23, into which the aspiration fluid 17 is guided.

A clamp 24 is also shown; it can be used to control the inflow of the treatment fluid 16 from the treatment fluid storage vessel 8 in the direction of the console 3. Furthermore, ports 18 are plotted, with which access may be provided to the treatment fluid storage vessel 8 or to the handpiece 2. A further valve 25 is also shown on the aspiration side 12.

It becomes clear that the treatment fluid 16 flows from the cassette 5 to the eye 15 through the handpiece 2. Moreover, aspiration fluid 17 is aspirated from the eye 15 in the direction of the cassette 5. The tubes used to this end for the treatment fluid 16 and the aspiration fluid 17 together form the supply device 10. Within the handpiece 2, the treatment fluid 16 may be guided through a sleeve 87 which surrounds a hollow needle 88 through which the aspiration fluid 17 is suctioned.

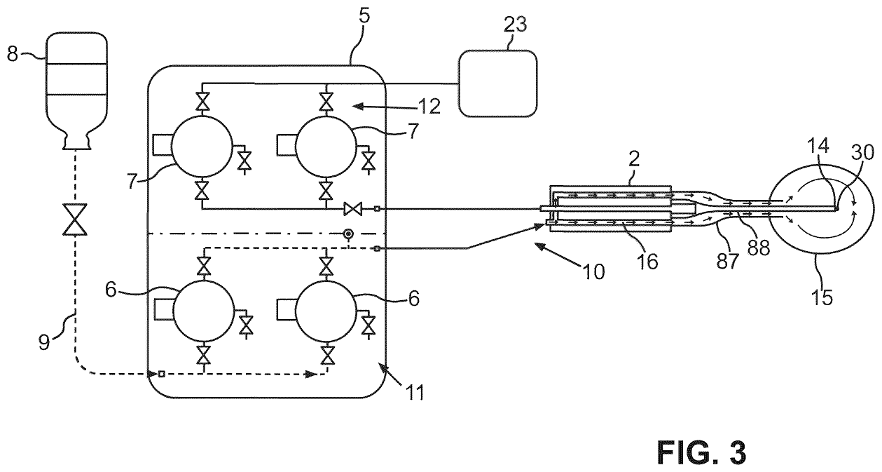

FIG. 3 shows the same construction as in FIG. 2, albeit for the case that a lens particle 30 blocks the needle tip 14 of the handpiece 2, with the result that the aspiration fluid 17 is no longer able to be aspirated from the eye 15. However, it is still possible to supply treatment fluid 16. This situation is referred to as an occlusion.

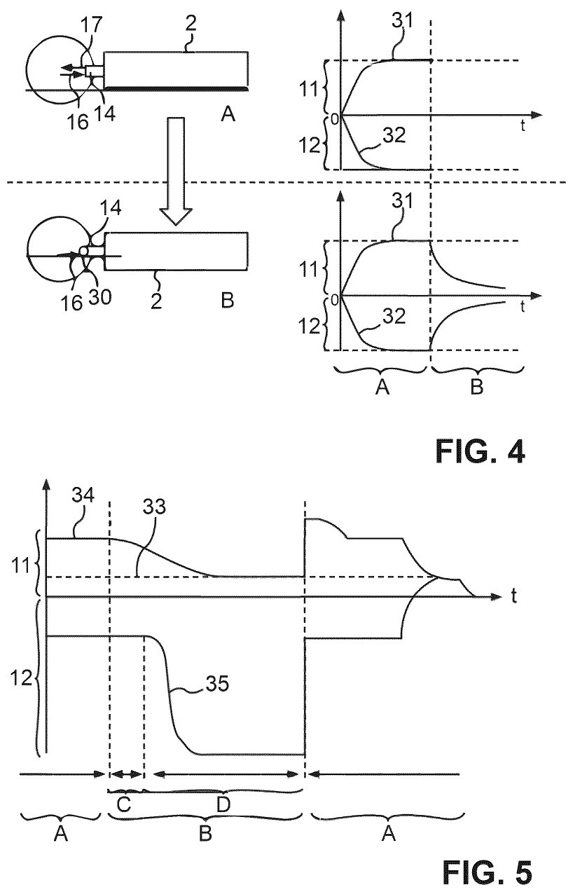

FIG. 4 describes a typical flow rate 31, 32 in relation to a time t preceding the occlusion, referred to here as situation A, and during the occlusion, referred to here as situation B. Before occlusion, the treatment fluid 16 can flow into the eye 15 and the aspiration fluid 17 can be aspirated from the eye 15. In that case, for example, a predetermined target value, plotted using a dashed line, is set in accordance with a first flow rate 31 on the irrigation side 11 and the discharge of aspiration fluid 17 up to the predetermined target value is shown on the aspiration side 12, in accordance with a second flow rate 32. For example, it is not possible to pump out any aspiration fluid 17 during the occlusion, and moreover the supply of further treatment fluid 16 is prevented. Thereupon, following the detection of the occlusion, both the first flow rate 31 on the irrigation side 11 and the second flow rate 32 on the aspiration side 12 reduce, in each case approaching 0.

FIG. 5 shows a profile of a set fluid pump pressure or a control air pressure p (y-axis) before the occlusion in situation A, during the occlusion in situation B, and also after the end of the occlusion in a new situation A. In this case, a first pressure profile 34 on the irrigation side 11, i.e., a fluid pump pressure profile or control air pressure profile on the irrigation side 11, and a second pressure profile 35 on the aspiration side 12, i.e., a fluid pump pressure profile or control air pressure profile on the aspiration side 12, are shown. Further, the occlusion in situation B is divided into a partial occlusion C and a full occlusion D. It becomes clear that a pressure in the irrigation-side fluid pump 6 is reduced on the irrigation side 11 during the partial occlusion C until a target pressure 33 is set in the eye 15. This is intended to prevent an undesirably strong increase in the pressure in the eye 15. The target pressure 33 should be maintained during the full occlusion D. Following the occlusion break, there is a strong, sudden increase in the pressure on the irrigation side 11 in order to compensate for a pressure drop in the eye 15 on account of a high aspiration-side pressure increase, until the target pressure 33 is reached again in the long term.

On the aspiration side 12, the aspiration fluid 17 is still discharged during the partial occlusion C, and the pressure at the aspiration-side fluid pump 7 undergoes a sudden drop in the case of the full occlusion D, when aspiration fluid 17 can no longer be pumped away. Following the occlusion break, the pressure at the aspiration-side fluid pump 7 increases again until it matches the target pressure 33. This is because the lens particle 30 no longer blocks the aspiration line and is sucked in after the occlusion break. As a consequence thereof, there is a sudden pressure increase at the aspiration-side fluid pump 7 on account of aspiration fluid 17 flowing into the aspiration line.

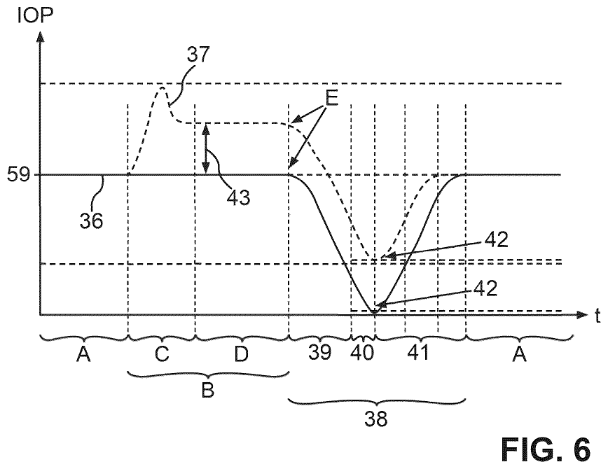

FIG. 6 shows a typical temporal profile of an intraocular pressure (IOP) in the eye 15 of a patient, referred to as standard profile 36, and a target profile 37 provided according to an exemplary embodiment of the disclosure. It becomes clear that initially before the occlusion in situation A, the standard profile 36 provides for the intraocular pressure to be set to a predetermined target intraocular pressure 59, which is patient specific, for example. Typically, attempts are made to maintain the target intraocular pressure 59, or at least to regulate back to said target intraocular pressure, during the occlusion and hence in situation B. The increase in the intraocular pressure at the start of the occlusion in situation B, shown for the target profile 37, is thus typically reduced in such a way that the predetermined target intraocular pressure 59 is reached again. Starting from the occlusion break, plotted here as a situation E, a particularly low intraocular pressure is achieved in the standard profile 36 since a large amount of aspiration fluid 17 is suddenly aspirated quickly from the eye 15 on the aspiration side 12. In this case, the target intraocular pressure 59 is again reached once a time period 38, composed of a sensory detection time 39 for detecting the occlusion break, a delay time 40 of an occlusion break detection algorithm and a response time 41 of the irrigation side 11 to the occlusion break for example, has elapsed.

The disclosure described below is intended to ensure that the target profile 37 is set. In this case, a minimum value 42 of the standard profile 36 is increased to the minimum value 42 of the target profile 37. Overall, a lower drop of the intraocular pressure following the occlusion break is observed as result, when compared with the standard profile 36. This is achieved by virtue of a target intraocular pressure increase 43 being determined and taken into account.

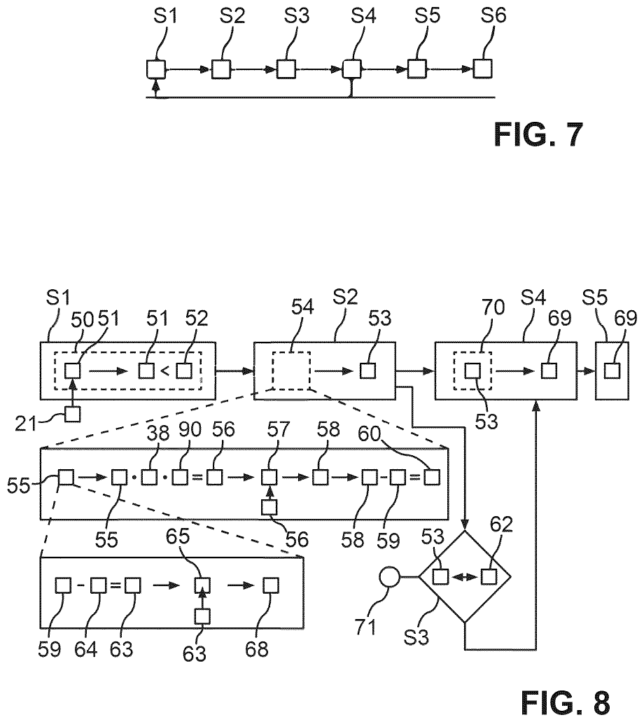

FIG. 7 shows the main steps which should be performed by the ophthalmic surgical system 1. FIG. 8 shows details of these main steps. The ophthalmic surgical system 1 is configured to detect an occlusion at the needle tip 14 in a method step S1. In a method step S2, it is configured, following the detection of the occlusion B, to determine intraocular pressure increase information 53. The latter describes the target intraocular pressure increase 43 by which the predetermined target intraocular pressure 59 of the eye 15 to be treated should be increased. To this end, an increase determination algorithm 54 is applied in method step S2, whereby the determined intraocular pressure increase information 53 depends on a current treatment situation.

The ophthalmic surgical system 1 is configured to check, in a method step S3, whether the determined intraocular pressure increase information 53 lies within a predetermined intraocular pressure value range 62. A control command 69 for the at least one irrigation-side fluid pump 6 is determined should this be the case, for example in a method step S4. To this end, the ophthalmic surgical system 1 in an example is configured to apply a command determination algorithm 70 to the determined intraocular pressure increase information 53. The determined control command 69 specifies an increase in the predetermined target intraocular pressure 59 by the target intraocular pressure increase 43 as a result of supplying treatment fluid 16 into the eye 15. Should the determined intraocular pressure increase information 53 be ascertained as lying outside of the predetermined intraocular pressure value range 62 in the method step S3, the method may be terminated 71 in the shown example.

In a method step S5, the ophthalmic surgical system 1 may be configured to control the at least one irrigation-side fluid pump 6 by applying an occlusion break detection algorithm in accordance with the determined control command 69 at least until the occlusion break of the detected occlusion is detected. The occlusion break may also be detected in a method step S6, whereupon there may be a switch to a normal operation of the ophthalmic surgical system 1, which was applied before the method step S1.

In detail, method step S1 may provide for the occlusion detection algorithm 50 to be applied to movement information 51 in order to detect the occlusion. Said movement information describes a current movement of the separation element 82, for example of the aspiration-side fluid pump 7. For example, the occlusion is detected at least on the basis of the movement information 51 being smaller than a predetermined first movement limit value 52. For example, the movement information 51 may be determined and provided by the position sensor 21.

In this case, the increase determination algorithm 54 is initially applied in method step S2. Furthermore, details regarding the workings of the increase determination algorithm 54 are shown. In an example and at a first level of detail, the following is carried out in the process: Initially, an expected volume flow 55 of aspiration fluid 17 is determined, in accordance with which it is expected that aspiration fluid 17 will be discharged from the eye 15 with the at least one aspiration-side fluid pump 7 after the occlusion break. It is initially assumed that the determined expected volume flow 55 is used to determine a target anterior chamber volume increase 56 in the eye, which describes a desired anterior chamber volume increase in the eye before the occlusion break in relation to an anterior chamber volume in the eye of an anterior chamber in the eye 15 before the occlusion. Here, it is also possible to take account of the predetermined time period 38 that is required for the detection of the occlusion break and a response to the occlusion break. In detail, the time period 38 may be composed of the detection time 39, the delay time 40 and the response time 41. For example, the expected volume flow 55 may be multiplied by the predetermined time period 38 in order to determine the target anterior chamber volume increase 56 in the eye. Moreover, a safety factor 90 may be taken into account in this multiplication as a further factor.

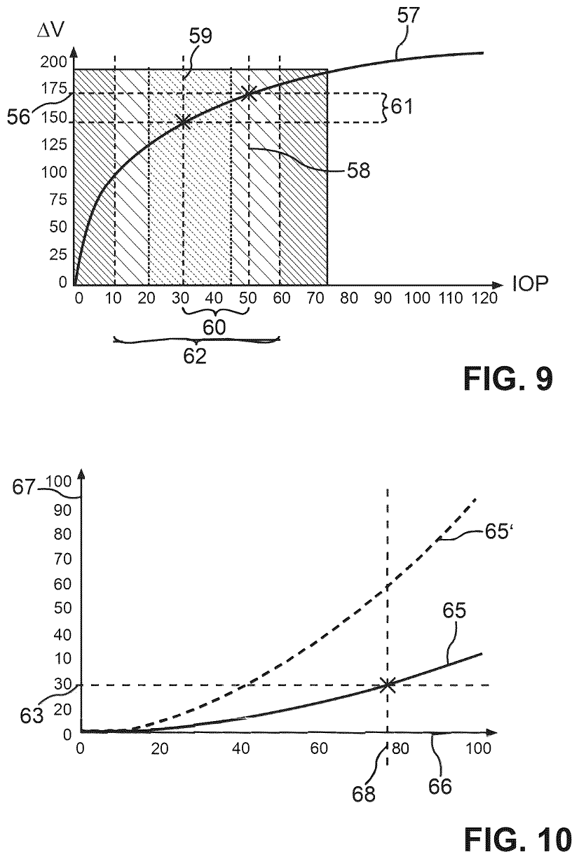

Furthermore, provision may then be made for an intraocular pressure 58 assigned to the determined target anterior chamber volume increase 56 in the eye to be determined using a predetermined correspondence curve 57, which is shown in FIG. 9 by way of example and describes a relationship between the anterior chamber volume increase in the eye and the intraocular pressure. Thereupon, an intraocular pressure difference 60 is determined between the predetermined target intraocular pressure 59 during the treatment of the eye 15 and the determined assigned intraocular pressure 58. In that case, the intraocular pressure difference 60 corresponds to the target intraocular pressure increase 43 in accordance with the intraocular pressure increase information 53.

Furthermore, a pressure difference 63 between the predetermined target intraocular pressure 59 and a current negative pressure 64 in the at least one aspiration-side fluid pump 7 may initially be determined in order to determine the expected volume flow 55. An assigned volume flow 68, which is assigned to the pressure drop corresponding to the determined pressure difference 63, is determined from a predetermined second correspondence curve 65, which is shown in FIG. 10 by way of example and describes a relationship between the volume flow 66 and an aspiration-side pressure drop 67, in particular in a manner specific to the handpiece 2. In that case, the determined assigned volume flow 68 corresponds to the expected volume flow 55.

For example, a check may be carried out in method step S3 as to whether the target intraocular pressure increase 43 in accordance with the determined intraocular pressure increase information 53 lies within the predetermined intraocular pressure value range 62 (FIG. 9). Method step S4 is carried out should this be the case.

FIG. 9 shows an example of the correspondence curve 57. Here, the intraocular pressure IOP is plotted on the x-axis, and the anterior chamber volume increase in the eye, denoted here as ΔV, is plotted on the y-axis. The anterior chamber volume increase in the eye is specified in microlitres, and the intraocular pressure is specified in millimetres of mercury (mmHg).

The predetermined target intraocular pressure 59 is plotted. In this case, the latter corresponds to a specific anterior chamber volume increase in the eye. In this context, it becomes clear that, for example for the target anterior chamber volume increase 56 in the eye, the associated value on the correspondence curve 57 can be read off, the latter then corresponding to the assigned intraocular pressure 58. The intraocular pressure difference 60 is accentuated, as is an anterior chamber volume change 61 in the eye, which corresponds to the difference between the target anterior chamber volume increase 56 in the eye and the anterior chamber volume increase in the eye assigned to the target intraocular pressure 59. Furthermore, the intraocular pressure value range 62 is shown between a minimum value and a maximum value. From left to right, the individual hatched regions describe an excessively low intraocular pressure, a permissible range for the intraocular pressure, a target range around the target intraocular pressure 59 followed by the permissible range and a range of excessively high intraocular pressure, reached here at 60 mmHg by way of example.

In an example, the predetermined correspondence curve 57 may be a conformity curve or a patient curve, which was determined specifically for a patient, or a group curve, which is predetermined for a patient group defined in particular depending at least on a sex, an age and/or an ethnicity. Alternatively, the conformity curve may be referred to as compliance curve.

FIG. 10 shows an example of the further correspondence curve 65. The latter is plotted here for two different handpieces 2, and so further correspondence curves 65, 65′ are shown here. In this case, the further correspondence curve 65′ is assigned to a handpiece 2 having a needle tip 14 with a smaller diameter than that associated with the further correspondence curve 65. Here, the x-axis plots the volume flow 66 at the aspiration-side fluid pump 7 in millilitres per minute and the y-axis plots the pressure drop 67 in mmHg. Now, the associated point of intersection of the for example further correspondence curve 65, which then corresponds to the determined assigned volume flow 68, is determined for the pressure difference 63.

FIG. 11 shows a detailed illustration of a fluid pump 6, 7, for example the aspiration-side fluid pump 7. It becomes clear that the fluid pump 6, 7 includes a pump chamber 80 and a drive chamber 81. The drive chamber 81 is coupled to the compressed-air valve 22. The position sensor 21 can be used to detect a movement of the separation element 82 between the pump chamber 80 and the drive chamber 81, for which purpose for example a metal plate 83 is positioned in the separation element 82 and its movement is detected with the position sensor 21. For example, the change in movement is accentuated here as deflection 85. In an alternative, the position sensor 21 may be arranged in surroundings of the drive chamber or compressed-air valve 22, i.e., on an opposite side of the fluid pump 6, 7 to what is shown here.

To detect the occlusion break, the occlusion break detection algorithm may be applied to second movement information 51. The occlusion break may at least be detected by virtue of the movement information 51 being larger than a predetermined second movement limit value 52. The second movement limit value 52 may be different from the first movement limit value 52 or correspond thereto.

Overall, the examples show pressure drop prevention by way of an adaptive and dynamic IOP increase following a detection of an occlusion. In his case, pressure drop means the pronounced, fast fall in intraocular pressure after the occlusion break. The disclosure shows how the response of the fluidic system can be controlled or regulated algorithmically in order to minimize the extent of pressure drop and enable a more health-preserving course of an operation for the patient. As a result of better maintenance of the IOP, the anterior chamber volume in the eye can be kept more stable, and the stress for the optic nerve and endothelial cells and anterior chamber collapse in the eye can be prevented. In this case, one possible solution is a dynamic increase in the intraocular pressure and hence an increase in the anterior chamber volume in the eye immediately after detection of the occlusion but before it breaks. However, in this context it should be observed that the IOP increase should always be kept as small as possible (because of endothelial cells, optic nerve; see explanations above) but as large as necessary in order to prevent a collapse of the anterior chamber in the eye as a result of the pressure drop owing to an occlusion break.

The aspiration-side fluid path between the eye 15 and pump diaphragms (fluid pump 7) can be illustrated on the basis of individual pressure losses of the components (needle tip 14, handpiece 2, aspiration line) in the aspiration-side fluid path between the eye 15 and pump diaphragm (fluid pump 7). Owing to their series connection, these may be combined as a total pressure loss, which only has a volume flow dependence. This total pressure loss is later required for feedforward control of the adaptive IOP adaptation and is referred to hereinafter as the aspiration characteristic line.

A possible solution for reducing the pressure drop after the occlusion break is described below, and so the pressure drop can be channelled by preventive measures even before the actual detection time 39 has elapsed. Thus, the extent of the pressure drop or the drop of the IOP below the target intraocular pressure 59 can be reduced, and critical IOP ranges may be avoided entirely.

The disclosure includes increasing the volume of the anterior chamber in the eye following an occlusion detection by precisely that value that is aspirated from the anterior chamber in the eye by the aspiration-side vacuum (negative pressure 64) in the time period 38 between the occlusion break, its detection and the initiation of countermeasures (as described above). The increase in the anterior chamber volume in the eye by increasing the IOP follows in accordance with the experimentally determined compliance curve (conformity curve) or the predetermined correspondence curve 57 in FIG. 9. Starting from the known detection and response time, which have only a negligible dependence on the aspiration-side and irrigation-side pressure losses, and the IOP target (target intraocular pressure 59) set at the user interface 13 for known needle tip pressure losses and vacuum suction pressure at the fluid pump 7, there is a calculation regarding the amount by which the IOP must be increased during the occlusion. A safety factor 90, which can be set by the user at the user interface 13, reduces the safety volume up or down and thus enables the attenuation of the pre-pressure drop IOP increase or the target intraocular pressure increase 43. For example, this may be recorded by a slider on the user interface 13. Moreover, it is possible to take into account a minimum and maximum value, by which the IOP is increased to a minimum and maximum extent in any case during an occlusion (intraocular pressure value range 62). The compliance curve stored in the algorithm as standard originates from literature data and reproduces the “standard eye”. In a further exemplary embodiment of the disclosure, it is possible for patient-specific compliance curves to be stored, and these may be determined prior to the treatment of the eye 15 or by clinical data. Alternatively, it is possible to store standard compliance curves for certain age groups or groups of origin (e.g.: Asian patient, female, age between 40 and 50 years). The background to this is found in particular in the fact that depending on ethnic origin and age, there is a change in the physical properties of the anterior chamber in the eye, and this thus has an influence on the relationship between anterior chamber volume in the eye and intraocular pressure. The data could be loaded as cloud data.

Initially there may be monitoring to check whether there is an occlusion and whether the pre-pressure drop adaptation function has been activated. This may be stored in the ophthalmic surgical system 1 by the user with the user interface 13 or by the manufacturer.

The following steps are then carried out, in particular with reference to FIG. 8:
1. Initially, the handpiece 2 used (taken from the user interface 13 or identified by the ophthalmic surgical system 1) is used to select the stored pressure loss curve (further correspondence curve 65) of the aspiration-side fluid path in the parameter set. The pressure discrepancy (pressure difference 63) driving the mass flow in the event of an occlusion break is calculated on the basis of the predetermined target intraocular pressure 59 and the aspiration negative pressure (negative pressure 64) at the aspiration pump diaphragm (aspiration-side fluid pump 7). Using the pressure discrepancy (pressure difference 63), the setting in volume flow 55 is determined on the basis of the characteristic map (further correspondence curve 65).
2. The volume flow 55 is multiplied by the detection and response time (time period 38) and a safety or attenuation factor (safety factor 90). Although the multiplication by the safety factor 90 may be stored in the function algorithm, it need not be visible on the user interface 13 or parametrized as 1 in the model. The multiplication yields the fluid volume flowing from the eye 15, i.e., the assigned volume flow 68 or the expected volume flow 55.
3. The compliance curve (correspondence curve 57) is determined using the set intraocular pressure target (target intraocular pressure 59) and a selection of stored patient data or clinical data. Said curve links an anterior chamber volume increase in the eye to an increase in the IOP and vice versa. Starting from the target intraocular pressure 59 set before the occlusion, the current anterior chamber volume in the eye is initially determined. The anterior chamber target volume in the eye is determined from the fluid volume to be additionally retained beforehand in the anterior chamber in the eye for the occlusion break (expected volume flow 55). The required pre-pressure drop IOP (assigned intraocular pressure 58) is determined from the characteristic map or compliance curve.
4. The pre-pressure drop IOP (assigned intraocular pressure 58) is subsequently still limited. The determined IOP may be limited in terms of its minimum value and its maximum value by the “upper limit” and “lower limit” parameters. In this case, the parameter set may be chosen on the basis of the handpiece 2 used, the eye or patient type or depending on other boundary conditions. A selection at the user interface 13 is also conceivable.

For example, the background for the two parameters is the following:
a. Lower limit: It is possible to choose a minimum pre-pressure drop IOP increase or the minimum target intraocular pressure increase 43 as a safety feature, for example in order to completely rule out an anterior chamber collapse in the eye in the case of a certain needle tip size.
b. Upper limit: An increase should be restricted on account of the endothelial cells and/or the optic nerve.
c. If both parameters are made the same using a specific value, then it is possible to choose a constant pre-pressure drop IOP increase independently of the feedforward control characteristic map.

The described method may be repeatedly carried out iteratively between method steps S1 and S4, since the driving pressure discrepancy increases as a result of an elevated IOP, and there may be a (slightly) higher volume flow after the occlusion break. In this case, the target intraocular pressure 59 is equated to the internal “IOP set value” parameter. An iterative calculation requires an increased (numerical) computational outlay. It is also possible to increase the safety factor 90 to 1.1 for example, especially in view of the possible short time scales between occlusion detection and occlusion break and, accompanying this, the avoidance of calculation-time-consuming iteration loops.

It was found that the pressure drop time and the recovery time, i.e., the time period 38, can be significantly reduced. Further advantages of the disclosure are: The extent of the pressure drop and the pressure drop duration may be reduced by up to 90 percent. Critical eye states may be reduced. The patient safety is increased. Needle tips 14 with a larger internal diameter and hence lower fluidic pressure losses may be rendered functional without software and parameter adaptation. During regular surgical operation, needle tips 14 with a large internal diameter have very good fluidic behaviour (holdability, followability) and a high suction force but a poor pressure drop behaviour on account of the low aspiration-side pressure losses. As a result of the dynamic IOP increase in the event of an occlusion, the problematic pressure drop behaviour during the operation of this needle tip 14 can be compensated for. The attenuation of the pressure drop is no longer subjected to the detection time 39. Channelling may be implemented at a higher IOP and prevents critical IOP states during the treatment. This can be implemented with state-of-the-art hardware by merely running a software update.

### LIST OF REFERENCE NUMERALS

1 Ophthalmic surgical system
2 Handpiece
3 Console
4 Cassette accommodation region
5 Cassette
6 Irrigation-side fluid pump
7 Aspiration-side fluid pump
8 Treatment fluid storage vessel
9 Treatment fluid line
10 Supply device
11 Irrigation side
12 Aspiration side
13 User interface
14 Needle tip
15 Eye
16 Treatment fluid
17 Aspiration fluid
18 Port
19 Inlet valve
20 Outlet valve
21 Position sensor
22 Compressed-air valve
23 Aspiration fluid collection vessel
24 Clamp
25 Valve
26 Pressure sensor
30 Lens particle
31 First flow rate
32 Second flow rate
33 Target pressure
34 First pressure profile
35 Second pressure profile
36 Standard profile
37 Target profile
38 Time period
39 Detection time
40 Delay time
41 Response time
42 Minimum value
43 Target intraocular pressure increase
50 Occlusion detection algorithm
51 Movement information
52 Movement limit value
53 Intraocular pressure increase information
54 Increase determination algorithm
55 Expected volume flow
56 Target anterior chamber volume increase in the eye
57 Correspondence curve
58 Assigned intraocular pressure
59 Target intraocular pressure
60 Intraocular pressure difference
62 Intraocular pressure value range
63 Pressure difference
64 Negative pressure
65, 65′ Further correspondence curve
66 Volume flow
67 Pressure drop
68 Assigned volume flow
69 Control command
70 Command determination algorithm
71 Termination
80 Pump chamber
81 Drive chamber
82 Separation element
83 Metal plate
85 Deflection
87 Sleeve
88 Hollow needle
90 Safety factor

What is claimed is:

1. An ophthalmic surgical system for treating an eye, the ophthalmic surgical system comprising:
a console including at least one portion of at least one irrigation-side fluid pump configured to convey a treatment fluid and at least one portion of at least one aspiration-side fluid pump configured to convey an aspiration fluid; and
an ophthalmic surgical handpiece configured to treat the eye, having a needle tip, at least a portion of the ophthalmic surgical handpiece being introducible into the eye with the needle tip of the handpiece, and being coupled at least indirectly to the irrigation-side fluid pump and the aspiration-side fluid pump,
wherein the ophthalmic surgical system is configured to:
detect an occlusion at the needle tip by applying an occlusion detection algorithm,
determine intraocular pressure increase information following the detection of the occlusion, the information describing a target intraocular pressure increase by which a predetermined target intraocular pressure in the eye to be treated should be increased, by applying an increase determination algorithm, wherein the determined intraocular pressure increase information depends on a current treatment situation,
determine a control command for the at least one irrigation-side fluid pump by applying a command determination algorithm to the determined intraocular pressure increase information, wherein the control command predetermines an increase in a current intraocular pressure to a sum of the target intraocular pressure and the target intraocular pressure increase by supplying treatment fluid into the eye, and
control the least one irrigation-side fluid pump at least until an occlusion break of the occlusion is detected by applying an occlusion break detection algorithm in accordance with the control command.

2. The ophthalmic surgical system according to claim 1, wherein, by applying the increase determination algorithm, the ophthalmic surgical system is configured to:
determine an expected volume flow of aspiration fluid, in accordance with which aspiration fluid is expected to be discharged from the eye with the at least one aspiration-side fluid pump after the occlusion break,
determine a target anterior chamber volume increase in the eye taking into account the expected volume flow, the target anterior chamber volume increase describing a desired anterior chamber volume increase in the eye before the occlusion break in relation to an anterior chamber volume in the eye of an anterior chamber in the eye before the occlusion,
determine an intraocular pressure assigned to the target anterior chamber volume increase in the eye with a predetermined correspondence curve which describes a relationship between the anterior chamber volume increase in the eye and the intraocular pressure, and
determine an intraocular pressure difference between a predetermined target intraocular pressure during the treatment of the eye and the assigned intraocular pressure, wherein the intraocular pressure difference describes the target intraocular pressure increase.

3. The ophthalmic surgical system according to claim 2, wherein, to determine the expected volume flow, the ophthalmic surgical system is further configured to:
determine a pressure difference between the predetermined target intraocular pressure and a current negative pressure in the at least one aspiration-side fluid pump, and
determine an assigned volume flow assigned to the pressure drop corresponding to the determined pressure difference, the assigned volume flow describing the expected volume flow, with a predetermined further correspondence curve which describes a relationship between a volume flow and an aspiration-side pressure drop for the handpiece.

4. The ophthalmic surgical system according to claim 2, wherein, in order to determine the target anterior chamber volume increase in the eye, the ophthalmic surgical system is further configured to take into account, in addition to the determined expected volume flow, a predetermined time period required for detecting the occlusion break and a response of the ophthalmic surgical system to the occlusion break to multiply the determined expected volume flow by the predetermined time period, wherein the product obtained in this case describes the target anterior chamber volume increase in the eye.

5. The ophthalmic surgical system according to claim 2, further configured to take into account a predetermined safety factor to determine the target anterior chamber volume increase in the eye.

6. The ophthalmic surgical system according to claim 2, further configured to adopt a conformity curve or a patient curve determined in a patient-specific manner or a group curve, which is predetermined for a patient group and defined based on at least a sex, an age, and/or an ethnicity, as the predetermined correspondence curve which describes the relationship between the anterior chamber volume increase in the eye and the intraocular pressure.

7. The ophthalmic surgical system according to claim 1, further configured to determine new intraocular pressure increase information at least once after the control of the at least one irrigation-side fluid pump in accordance with the control command and before the occlusion break is detected, with this taking account of a target intraocular pressure adapted to a response time of the eye to a change in the intraocular pressure, to determine a new control command by applying the command determination algorithm to the new intraocular pressure increase information and to control the at least one irrigation-side fluid pump at least until the detection of the occlusion break in accordance with the new actuation command.

8. The ophthalmic surgical system according to claim 1, further configured to follow the determination of the intraocular pressure increase information with carrying out a check as to whether the target intraocular pressure increase in accordance with the determined intraocular pressure increase information lies in a predetermined intraocular pressure value range, wherein the control command is determined when the check is resolved in the affirmative.

9. The ophthalmic surgical system according to claim 1, further configured to:
apply the occlusion detection algorithm to movement information describing a current movement of a separation element between a pump chamber and a drive chamber of at least one of the fluid pumps to detect the occlusion, and detect the occlusion at least on the basis of the movement information being smaller than a predetermined first movement limit value, and/or
apply the occlusion break detection algorithm to the movement information to detect the occlusion break, and detect the occlusion break at least based on the movement information being larger than a predetermined second movement limit value.

10. A method for operating an ophthalmic surgical system having at least a console which includes at least one portion of at least one irrigation-side fluid pump configured to convey a treatment fluid and at least one portion of at least one aspiration-side fluid pump configured to convey an aspiration fluid, and an ophthalmic surgical handpiece having a needle tip, at least a portion of the ophthalmic surgical handpiece being introducible into an eye with the needle tip of the ophthalmic surgical handpiece, and the ophthalmic surgical handpiece being coupled at least indirectly to the irrigation-side fluid pump and the aspiration-side fluid pump, the method comprising:
determining intraocular pressure increase information following a detection of an occlusion at the needle tip, the information describing a target intraocular pressure increase by which a predetermined target intraocular pressure should be increased, by applying an increase determination algorithm, wherein the intraocular pressure increase information depends on a current treatment situation;
determining a control command for the at least one irrigation-side fluid pump by applying a command determination algorithm to the intraocular pressure increase information, wherein the control command predetermines an increase in a current intraocular pressure to a sum of the target intraocular pressure and the target intraocular pressure increase by supplying treatment fluid into the eye, and
controlling the least one irrigation-side fluid pump at least until an occlusion break of the detected occlusion is detected by applying an occlusion break detection algorithm in accordance with the control command.
